(19) United States
(12) Patent Application Publication
(10) Pub. No.: US 2025/0250726 A1
(43) Pub. Date: Aug. 7, 2025

(54) Method for Manufacturing an Absorption and Distribution Nonwoven Fabric

(71) Applicant: Sandler AG, Schwarzenbach (DE)

(72) Inventors: Uwe Bernhuber, Hof (DE); Veronika Trapp, Hof (DE)

(21) Appl. No.: 19/045,848

(22) Filed: Feb. 5, 2025

(30) Foreign Application Priority Data
Feb. 7, 2024 (DE) 10 2024 103 421.1

(51) Int. Cl. D04H 1/407; D04H 1/4258; D04H 1/435; D04H 1/4374; D04H 1/4382; D04H 1/485; D04H 1/498

(52) U.S. Cl. CPC D04H 1/407; D04H 1/4258; D04H 1/435; D04H 1/4374; D04H 1/43825; D04H 1/43835; D04H 1/485; D04H 1/498; D10B 2201/24; D10B 2401/022; D10B 2401/06; D10B 2509/026

(57) ABSTRACT

The invention relates to a method for manufacturing an absorption and distribution nonwoven fabric made of staple fibers and absorbent material made of regenerated cellulose for personal hygiene products. The nonwoven fabric is composed of thermoplastic, synthetic staple fibers as support fibers, wherein the support fibers are homo- or bi-component, thermoplastic regenerated cellulose for personal hygiene products. The nonwoven fabric is composed of thermoplastic, synthetic staple fibers as supporting fibers, wherein the supporting fibers are homo- or bi-component, thermoplastic polymer fibers comprising fusible constituents, staple fibers of thermoplastic and/or duroplastic polymers as distribution fibers and absorbent material of regenerated cellulose. The nonwoven fabric is mechanically bonded and then thermally bonded by means of subsequent hot-air bonding. The invention also relates to an absorption and distribution nonwoven fabric produced by the method according to the invention and to a device for carrying out the method according to the invention.

### CROSS-REFERENCE TO RELATED APPLICATION

This application claims the benefit of priority to German Patent Appl. No. 102024103421.1 filed Feb. 7, 2024, the disclosure of which is hereby incorporated by reference in its entirety.

### BACKGROUND

The present invention relates to a method for manufacturing an absorption and distribution nonwoven fabric for personal hygiene products according to the independent claim 1.

In a method according to the prior art, the nonwoven fabric is composed of thermoplastic, synthetic staple fibers as support fibers, wherein the support fibers are homo- or bi-component, thermoplastic polymer fibers that comprise fusible constituents, staple fibers made of thermoplastic and/or duroplastic polymers as distribution fibers and absorbent material made of regenerated cellulose, wherein the nonwoven fabric is mechanically bonded and then thermally bonded by means of subsequent thermal activation, for example by hot-air bonding.

A corresponding method is known from EP2692321B1. In this case, the mechanical bonding is carried by means of a water jet process. With this established method, nonwoven fabrics with acceptable absorption and distribution properties can be produced.

Nonwoven fabrics produced in this way are used as part of hygiene products, which in principle have the following components:

topsheet as the layer facing the body
an absorption and distribution layer for rapid absorption of the incoming liquid and distribution over the entire surface of the hygiene product
a storage layer to immobilize the amount of liquid
a backsheet as a liquid-tight outer layer

In recent years, the trend has been towards a storage layer consisting of superabsorbent polymers and cellulose. The upstream absorption and distribution layer therefore has the task of temporarily storing the liquid to prevent gel blocking of the superabsorbers used. The liquid must also be distributed over the entire surface of the hygiene article so that the storage layer is fully utilized.

Accordingly, there is still a need for a method of manufacturing an absorbent and distribution nonwoven fabric that comprises further improved absorbent and distribution properties and also has an improved intermediate storage capacity and improved cushioning properties.

The task was therefore to provide a method for manufacturing an absorption and distribution nonwoven that enables the provision of an improved absorption and distribution nonwoven with improved absorption and distribution properties, an improved intermediate storage capability and improved cushioning properties.

### SUMMARY

The problem is solved by the features of claim 1. Accordingly, the problem is solved in accordance with the invention if the nonwoven fabric is composed of 5 to 35 wt. % of the support fibers, 30 to 90 wt. % of the distribution fibers and 5 to 35 wt. % of the absorbent material and the mechanical bonding is carried out by means of needling.

By means of the corresponding composition of the fibers and the needling as a mechanical bonding method, an absorbent and distribution nonwoven with improved properties can be manufactured using the method according to the invention. In this way, it became apparent that an improved openness of the nonwoven fabric and thus the ability to quickly absorb incoming liquid and distribute it over the surface of the nonwoven fabric according to the invention is achieved because, due to the needling, the interlacing is only achieved selectively via individual needles. In addition, the stitch channels caused by the penetration of the needles surprisingly provide improved capillarity, which in particular improves the distribution properties of the nonwoven produced. Furthermore, an alignment in the Z-direction is achieved, which is beneficial for the material thickness. Thus, surprisingly, nonwoven fabrics with higher material thicknesses per weight per unit area can be produced using the method according to the invention, which is limited to a very narrow range between 0.12 mm-0.15 mm per 10 g/m² using the previously known method. The higher material thicknesses per weight per unit area can significantly improve the cushioning properties and the intermediate storage capacity of the absorption and distribution nonwoven produced by the method according to the invention.

### BRIEF DESCRIPTION OF THE DRAWINGS

FIG. 1 illustrates a formula for calculating the penetration density

FIG. 2 illustrates nomenclature of felting needles.

### DETAILED DESCRIPTION

The terms absorbent material, distribution fiber and support fiber as used in the invention are defined as follows:

Absorbent material is either a pulp or a regenerated cellulose fiber, which is produced from solutions of cellulose derivatives, whereby the cellulose fiber is preferred as absorbent material.

The pulp is a flaked material obtained by chemical pulping of wood, having a fiber length of 1 to 4 mm, and can be used as absorbent material.

Regenerated cellulose fibers according to the invention, such as viscose, may have a modified surface and/or cross-section. For example, a trilobal cross-section significantly increases absorbency compared to a round cross-section. A hollow viscose fiber may also be used.

Representatives according to the invention are, for example, Viscostar viscose fibers or normal viscose fibers from the manufacturer Lenzing, but also the so-called Galaxy fiber from Fa. Kelheim Fibers. The usual fiber titres are in the range of 1.0 to 3.3 dtex, preferably 1.3 to 2.2 dtex: the fiber lengths used are in the range of 10-70 mm, preferably 35-50 mm.

Furthermore, regenerated cellulose fibers can also be produced according to the invention using the so-called lyocell method, which comprise better stability, particularly when wet, so that the distribution of liquid is favored when liquid is absorbed to a moderate extent.

Representatives according to the invention are, for example, Lyocell or Tencel-viscose fiber from the company Lenzing. Usual fiber titres are in the range of 1.0 to 3.3 dtex, preferably 1.3 to 2.2 dtex; the fiber lengths used are in the range of 10-70 mm, preferably 35-50 mm.

Even when the aforementioned Lyocell or Tencel fibers are used, they cannot completely take over the task of liquid distribution: an admixture of polyester and/or polyacrylic fibers within the amount of distribution fibers used is possible.

In the context of the present invention, the term “distribution fiber” is used to refer to fibers made of thermoplastic and/or duroplastic polymers that ensure the transport of liquid in the nonwoven fabric according to the invention.

The fibers used for this purpose are preferably permanently hydrophilic fibers made of thermoplastic polymers such as polyester or polypropylene. However, duroplastic polymers such as polyacrylic fibers are also suitable.

The term hydrophilic refers to fibers which, in their unprocessed state, comprise a sink time of less than 5 seconds, preferably less than 3 seconds. The sinking time is determined in accordance with NWSP 010.1 R0 (20).

In addition to full fibers, i.e. fibers that consist entirely of polymer mass over their cross-section, hollow fibers can also be used. Hollow fibers comprise one or more cavities over the cross-section of a fiber, so that such fibers resemble a tube when viewed along their length. The fiber titres are in the range of 1.7 to 10 dtex, preferably 2.2 to 4.4 dtex: the fiber lengths used are in the range of 10-80 mm, preferably 35-60 mm. Furthermore, fibers that do not have a round fiber cross-section, e.g. trilobal, pentalobal or similar, are used for liquid distribution.

Support fibers according to the present invention are fibers that stabilize the fiber composite in a product manufactured according to the invention, so that a largely dimensionally stable nonwoven fabric is manufactured. Support fibers are homo- or bi-component, thermoplastic polymer fibers that comprise fusible constituents. When heated to their softening point, these constituents melt and stabilize the fiber composite after cooling. Fibers suitable for the invention are homopolymeric staple fibers made of co-polyester, co-polyamide or polypropylene: bicomponent melt fibers in core-sheath or side-by-side arrangement made of combinations of low-melting co-polyester with polyester, polyethylene with polypropylene or polyethylene with polyester are preferred for the invention. The fiber titres are in the range of 1.7 to 4.8 dtex, preferably 2.2 to 4.4 dtex; the fiber lengths used are in the range of 10-80 mm, preferably 35-60 mm.

Advantageous embodiments of the present invention are the subject of the dependent claims.

According to a further preferred embodiment, the fibers of the nonwoven fabric are homogeneously mixed with each other prior to mechanical bonding and formed into a fibrous web by means of a carding process. Preferably, the homogeneous mixing is carried out by means of bale opening and mixing devices.

In another particularly preferred embodiment of the present invention, the fibrous web is laid down in a longitudinal direction. This allows an absorption and distribution nonwoven fabric to be produced that comprises the necessary longitudinal stability for use in hygiene products.

In another preferred embodiment of the present invention, the fibrous web is laid down as a layered structure comprising a first layer and a second layer, wherein the first layer is laid in the longitudinal direction and the second layer is laid in the transversal direction or with an isotropic orientation and is laid on or under the first layer. This allows the production of an absorption and distribution nonwoven fabric that comprises the necessary longitudinal stability for use in hygiene products.

Preferably, the layered structure comprises two first layers, wherein the second layer is laid between the two first layers. An absorption and distribution nonwoven fabric can also be produced in this way, which comprises the necessary longitudinal stability for use in hygiene products. In addition, the absorption and distribution nonwoven fabrics produced have particularly advantageous cushioning properties.

Further preferably, the first layers each comprise 25-40% of the weight per unit area and the second layer comprises 20-50% of the weight per unit area. This improves the advantageous cushioning properties of the absorption and distribution nonwoven fabric produced.

According to a particularly preferred embodiment of the present invention, the needling is carried out with at least 200 active barbs/cm² of nonwoven fabric, preferably with 200 to 1000 active barbs/cm 2 of nonwoven fabric, more preferably with 300 to 600 active barbs/cm² of nonwoven fabric, and most preferably with 400 to 500 active barbs/cm² of nonwoven fabric. The active barbs/cm² of nonwoven fabric define the needling intensity. They are determined by the number of barbs per needle, the penetration depth and thus the active barbs, and the penetration density. For example, if the needles used comprise 6 barbs and 100 punctures/cm² of nonwoven fabric are made during needling with all barbs penetrating, this results in 600 active barbs/cm² of nonwoven fabric. Surprisingly, it has been shown that the claimed number of active barbs/cm² of nonwoven fabric results in improved liquid absorption and distribution properties of the nonwoven fabric, while the nonwoven fabric also has a good cushioning effect.

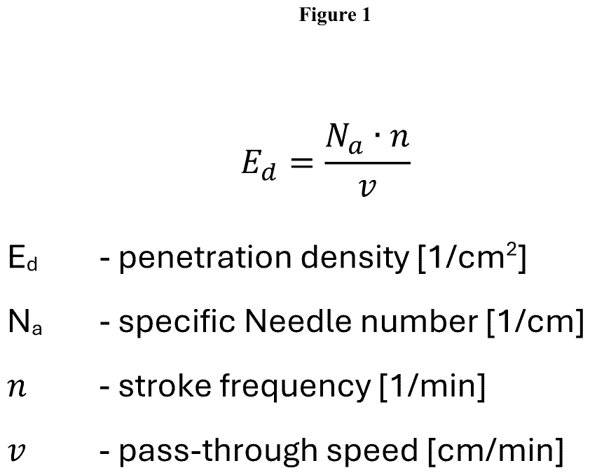

Formula for calculating the penetration density is shown in FIG. 1.

Furthermore, preferably, individual needles used for needling comprise between 1 and 9, preferably between 3 and 6, barbs. This number of barbs results in an advantageous interlacing in the nonwoven fabric.

Also preferably, the needles used for needling comprise a diameter between 30 and 46 gauge, preferably between 38 and 40 gauge of the working part of the needle, and a total length of 2 to 3 inches. The diameter is specified using the gauge system (Albrecht, Fuchs, Kittelmann: Vliesstoffe. Wiley-VCH Verlag, 2000, p.291). The cross-section of the working part and the arrangement and spacing of the barbs in this case are irrelevant. Surprisingly, it has been shown that the stitch channels caused by the use of such needles provide improved capillarity without any deterioration in liquid distribution.

In a further particularly preferred embodiment of the present invention, the needling is carried out with at least two, preferably four, needle boards, which penetrate the incoming nonwoven in alternating fashion on both sides, wherein the needle boards preferably comprise a needle density of 12,000 to 80,000 needles/m of working width, and more preferably 20,000 to 50,000 needles/m of working width. Needling on both sides can achieve a uniform capillarity of the nonwoven fabric produced, resulting in improved liquid distribution.

In another preferred embodiment, thermal activation is carried out by means of hot-air bonding, wherein the nonwoven fabric is heated to a temperature above the melting point of the fusible constituents of the support fibers, preferably for at least 20 seconds during the hot-air bonding. This results in an advantageous consolidation of the absorption and distribution nonwoven fabrics manufactured.

In another preferred embodiment, the thermal activation is carried out by means of infrared waves.

According to a preferred embodiment, the nonwoven fabric, after mechanical and thermal bonding, comprises a material thickness per weight per unit area of at least 0.20 mm/10 g/m², particularly preferably at least 0.25 mm/10 g/m². This allows the production of absorption and distribution nonwovens with particularly advantageous intermediate storage capabilities.

In another preferred embodiment, the nonwoven fabric is composed of 5 to 10 wt. % of the support fiber, 80 to 90 wt. % of the distribution fiber and 5 to 10 wt. % of the absorbent material. A nonwoven fabric produced in this way has particularly good distribution properties.

The invention is also directed to an absorption and distribution nonwoven fabric produced according to one of the above-described embodiments of the method according to the invention. The nonwoven fabric according to the invention comprises a material thickness, softness, stiffness, liquid distribution and liquid retention under load that is superior to the prior art.

According to a preferred embodiment, the nonwoven fabric has a material thickness per weight per unit area of at least 0.20 mm/10 g/m², and is particularly preferred to have a thickness of at least 0.25 mm/10 g/m². As a result, the absorption and distribution nonwoven fabric has particularly advantageous intermediate storage capabilities.

According to a preferred embodiment of the invention, the absorption and distribution nonwoven fabric comprises a weight per unit area of 40 to 150 g/m², preferably between 50-100 g/m². Such absorption and distribution nonwoven fabrics are particularly suitable for use in hygiene products.

According to a further preferred embodiment of the invention, the absorption and distribution nonwoven fabric comprises at least a Run In, measured according to the method described below, of 10%, preferably of 15%. Such a nonwoven fabric has a preferred intermediate storage capacity.

According to a further preferred embodiment of the absorption and distribution nonwoven fabric according to the invention, the nonwoven fabric comprises at least a Quantitative Absorption vertically after 20 seconds, measured according to the method described below, of 1500%, preferably of 2000%. Such a nonwoven fabric has a preferred absorption capacity.

According to a further preferred embodiment of the absorption and distribution nonwoven fabric according to the invention, the nonwoven fabric comprises a compression hardness 40%-according to DIN ISO 3386-1, determined in the first load cycle—of at most 15 N, preferably at most 9 N. Such a nonwoven fabric has an improved cushioning effect.

In addition, the invention is directed to a device for carrying out a method according to one of the above-described embodiments of the method according to the invention.

Two methods for manufacturing absorption and distribution nonwoven fabrics according to the invention and the absorption and distribution nonwoven fabrics manufactured thereby are described below and compared with a previously known method for manufacturing an absorption and distribution nonwoven fabric and the absorption and distribution nonwoven fabric manufactured thereby.

The underlying process steps, such as mechanical needling, water jet processing or thermal hot-air bonding, can be found in the book “Vliesstoffe”, published by Wiley-VCH, Weinheim, in 2000.

### Manufacturing Method According to the Prior Art

An exemplary manufacturing method according to the prior art comprises providing a mixture of
→60% distribution fibers made of hydrophilic polyethylene terephthalate fibers with a titer of 3.3 dtex and a staple length of 38 mm.
→25% absorbent material, formed from a commercially available hydrophilic viscose fiber with a titer of 1.7 dtex and a staple length of 42 mm
>15% support fibers, formed from a hydrophilic polyethylene terephthalate/co-polyethylene terephthalate fiber with a titer of 4.4 dtex and a staple length of 51 mm

The fibers are homogeneously mixed with each other and formed into a fibrous web by means of a carding process, which is laid down in the longitudinal direction.

The fibrous web is then mechanically bonded on both sides by means of a water jet process with a double-row nozzle strip with 40 hpi and a nozzle hole diameter of 0.12 mm at cumulative pressures of the nozzle bars between 400 and 700 bar, and then thermally bonded by means of subsequent hot-air bonding.

A corresponding nonwoven is available from Sandler AG under the product name Sawasoft® 1236-50 g/m².

Method according to the invention for manufacturing a nonwoven 1:
For the first method for manufacturing a nonwoven according to the invention, a fiber mixture consisting of
→60% distribution fibers made of hydrophilic polyethylene terephthalate fibers with a titer of 3.3 dtex and a staple length of 38 mm,
→25% absorbent material, formed from a commercially available hydrophilic viscose fiber with a titer of 1.7 dtex and a staple length of 42 mm, and
→15% support fibers, formed from a hydrophilic polyethylene terephthalate/co-polyethylene terephthalate fiber with a titer of 4.4 dtex and a staple length of 51 mm is used.

The fibers are homogeneously mixed with each other and formed into a fibrous web by means of a carding process, which is laid down in the longitudinal direction.

This fibrous web then first passes through a needle loom of the type Cyclopunch OUG-2 from the manufacturer Dilo. This so-called intensive needling passage is configured with four needle boards, each with 20183 needles per board and meter of working width, which penetrate the incoming web alternately on both sides. In total, all needle boards act on the incoming nonwoven with a needle density of 80732 needles per meter working width. The needle boards are equipped with a needle type 15×18×40×2.5 R222 V2206 from the manufacturer Groz-Beckert.

The nomenclature of felting needles is is shown in FIG. 2.

The designation R222 defines the number of barbs (here a total of 6)—an essential characteristic for needles. In the first method for manufacturing according to the invention, needling is carried out at 147 strokes/min and a feed of 53 mm in the machine direction per stroke. The penetration depth is 6.0 mm and the penetration density 150 E/cm², resulting in a needling intensity of 300 active barbs/cm².

The pre-consolidated nonwoven 1 is subjected to a subsequent heat treatment in the form of hot-air bonding to activate the fusible constituents within the support fibers present in accordance with the invention. Depending on the type of support fibers used, the nonwoven is heated to temperatures above the melting point of the fusible constituents of the support fibers. This temperature must be reached in the nonwoven for about 30 seconds to achieve sufficient melting flow of the fusible constituents. In the case of the first method for manufacturing according to the invention, the temperature was 150° C.

Finally, the product obtained in this way is cooled and can be processed as required for further processing. The nonwoven produced in this way can have a weight per unit area in the range from 40 to 150 g/m², preferably 50-90 g/m².

Method according to the invention for manufacturing a nonwoven 2:

For the second method for manufacturing according to the invention, a fiber mixture consisting of
→60% distribution fibers, including 35% hydrophilic polyethylene terephthalate fibers with a titer of 3.3 dtex and a staple length of 38 mm.
→25% absorbent material, formed from a commercially available hydrophilic viscose fiber with a titer of 1.7 dtex and a staple length of 42 mm
→15% support fibers, formed from a hydrophilic polyethylene terephthalate/co-polyethylene terephthalate fiber with a titer of 4.4 dtex and a staple length of 51 mm is used.

The fibers are also homogeneously mixed with each other as described in the first method for manufacturing according to the invention and formed into a fibrous web by means of a carding process. In this process, a layered structure is produced with the aid of at least three cards. The upper and lower layers (first layers) are carded and laid down in the longitudinal direction. The middle layer (second layer) is guided between the two longitudinally laid layers by cross-laying a card web using a standard cross-lapper of the Hyperlayer (HLSC) type from Dilo, resulting in a hybrid product. The middle layer has an isotropic fiber orientation, while the top and bottom layers are oriented in the longitudinal direction.

To produce this middle layer, a card web of approx. 30 g/m² is turned 90° to the fiber direction of the longitudinal nonwovens already described and first fed into the cross-lapper described above. This lays down a total of 4 individual layers, which are first stretched over a nonwoven drafting unit with 200% draft to 40 g/m² before converging with the two longitudinal nonwovens (bottom and top layer, each with a weight per unit area of 30 g/m²). A Dilo nonwoven drafting unit (type VST19) is used for the nonwoven drafting.

The so-called hybrid product thus consists of a 30 g/m² bottom layer, a 40 g/m² middle layer and a 30 g/m² top layer, which are brought together shortly before needling. The individual layers can vary in the mass fractions as follows:
Top and bottom layer: 25-40% of the weight per unit area
Middle layer: 20-50% of the weight per unit area

This fibrous web then also passes through a needle loom of the type Cyclopunch OUG-2 from the manufacturer Dilo, analogous to the first method for manufacturing according to the invention. This so-called intensive needling passage is also designed with four needle boards, each with 20,183 needles per board and meter of working width, which penetrate the incoming web alternately on both sides. In total, all needle boards have a needle density of 80,732 needles per meter of working width on the incoming nonwoven. The needles used can vary in shape depending on the desired intensity, which affects the material thickness. In this case, the needle boards are equipped with a needle type 15×18×40×2.5 R222 V2206 from the manufacturer Groz-Beckert. The designation R222 defines the number of barbs (here a total of 6), which is an essential characteristic of needles. In the second method for manufacturing according to the invention, needling is performed at 147 strokes/min and a feed of 53 mm in the machine direction per stroke. The penetration depth is 6.0 mm and the penetration density 150 E/cm², resulting in a needling intensity of 300 active barbs/cm².

The pre-consolidated nonwoven 2 is then subjected to a subsequent heat treatment in the form of hot-air bonding to activate the fusible constituents within the support fibers present in accordance with the invention. Depending on the type of support fibers used, the nonwoven is heated to temperatures above the melting point of the fusible constituents of the support fibers. This temperature must be reached in the nonwoven for about 30 seconds in order to achieve sufficient fusion of the fusible constituents. In the case of the second method for manufacturing according to the invention, the temperature was 150° C.

Finally, the product obtained in this way is cooled and can be processed as required for further processing. The nonwoven produced in this way can have a weight per unit area in the range from 40 to 150 g/m², preferably 50-90 g/m².

### Comparison of the Produced Nonwovens

The nonwovens produced according to the invention-nonwoven 1 and nonwoven 2—are compared below with the nonwoven from the prior art-Sawasoft® 1236.

The test results are shown in Table 1 below. The parameters determined were determined according to the test methods mentioned below:

→Weight per unit area according to DIN EN 29073-1
→Thickness according to DIN EN ISO 5084
→Thickness quotient: material thickness in mm per 10 g/m² of nonwoven fabric
→Tensile test according to DIN EN ISO 9073-03
→Absorption rate according to NWSP 010.1.R0 (20) (modified method described below)
→Run Off according to NWSP 80.9 R1 (19) (the additional modified method for this is described below)
→Compression hardness 40% according to DIN ISO 3386-1

### Description of the Measuring Method for the Run-In

The NWSP 080.9.R1 (19) test standard describes the nonwoven run-off. Run-off is defined as the amount of liquid that is not absorbed by the nonwoven test specimen and runs out at the lower end of the sample. This amount is weighed and set in relation to the amount of liquid applied. The details are described in the test standard.

In analogy to this test standard, a run-in is measured by changing the inclination of the draining table from 25° to 45°. This is defined as follows: the run-in is the amount of liquid that remains on the nonwoven sample 30 seconds after the liquid has been applied to it, i.e. the liquid that neither runs out nor is absorbed by the underlying absorption medium. The run-in is measured by removing the nonwoven from the inclined draining table and the underlying absorption medium after the defined waiting time of 30 s and weighing it. The weight corresponds to the total mass of the nonwoven and the liquid absorbed. After deducting the previously determined dry mass of the nonwoven sample, the amount of liquid absorbed is related to this dry mass. The calculation is done according to the following formula:

$$\text{Run-In} = ( {m}_{2} - {m}_{1} ) / {m}_{1}$$

where
m²=mass of the nonwoven sample with the absorbed amount of liquid after a 30-second waiting period
m¹=dry mass of the nonwoven test specimen

The run-in can be specified in g/g or in % of absorbed liquid. The run-in characterizes the intermediate storage capacity of a nonwoven fabric when exposed to a specific amount of liquid at a single point.

### Description of the Measurement Method for Capillary Liquid Absorption

The test standard NWSP 010.1.R10 (20) describes three test methods for nonwoven absorption, including the method of capillary liquid absorption known as rising height measurement, the implementation of which is described in chapter 8.3 of this standard.

In an extension of this measuring method, in addition to the rising heights, the masses of the capillary-absorbed liquid are determined after defined times. To do this, the storage vessel with the liquid from the capillary rise is placed on a scale so that after dipping the nonwoven sample, the liquid sucked in can be measured as a negative weight. To do this, the scale on which the vessel with the liquid is located is tared to zero before the sample is dipped in. After the test specimen has been immersed, the rising heights are read off after defined times, as described in the standard, and the weight displays on the balance are read in parallel. Since the liquid is sucked out of the storage vessel by the nonwoven fabric, the weight of the storage vessel decreases, i.e. the display shows negative values.

As the nonwoven fabric is wetted, a meniscus forms at the edge of the test specimen, starting from the liquid surface. The lamella of liquid that forms as a result of surface tension also pulls on the balance as a negative weight and must be taken into account. It can be determined approximately, but with sufficient precision for the purposes of interpreting the results, as follows: The test specimen is left in the liquid until the capillary rise no longer changes by a millimeter within three minutes, i.e. capillary equilibrium can practically be assumed. The nonwoven strip is then pulled out of the liquid, the balance is tared to zero and the nonwoven strip is moved to the surface until the liquid touches the nonwoven strip. At that moment, the liquid lamella jumps onto the nonwoven strip due to surface tension, without liquid being absorbed or released by the nonwoven. The display on the balance gives a good approximation of the weight of the liquid lamella, by which the values generated during the measurement are corrected.

The evaluation is done according to the following formula:

$$Capillary absorption = ( {m}_{t} - {m}_{L} ) / {m}_{V}$$

where
mₜ=weight read on the balance after time t m_{L}=weight of the liquid lamella, determined as described above m_{y}=mass of the wetted nonwoven zone, which is determined from the area determined on the basis of the capillary rise and the weight per unit area using the following formula:
m_{y}=h×b×FG where
h=capillary rise of the liquid
b=width of the nonwoven sample
FG=weight per unit area of the nonwoven fabric in g/m²

The mass-related capillary absorption gives a more realistic representation of the time-dependent liquid absorption than the capillary rise.

TABLE 1
Properties of the nonwovens according to
the invention compared to the prior art

| | | Sawasoft ® | | |
|---|---|---|---|---|
| | Test standard | 1236 | Vlies 1 | Vlies 2 |
| Weight per unit | DIN EN | 50 g/m² | 61.6 g/m² | 83.5 g/m² |
| area | 29073-1 | | | |
| Thickness at | DIN EN ISO | 0.61 mm | 1.93 mm | 2.40 mm |
| 0.5 kPa preload | 5084 | | | |
| Thickness | calculated | 0.12 mm | 0.31 mm | 0.29 mm |
| quotient | | per 10 | per 10 | per |
| | | g/m² | g/m² | 10 g/m² |
| Run Off | NWSP 80.9 R1 | 0% | 0% | 0% |
| | (19) | | | |
| Run In | In reference to | 4.4% | 19.4% | 15.4% |
| | NWSP 80.9 R1 | | | |
| | (19) | | | |
| Compression | DIN ISO | 17.3N | 8.9N | 7.3N |
| hardness 40% | 3386-1 | | | |
| Quantitative | In reference to | 720% | 3243% | 2071% |
| absorption | NWSP 010.1.R0 | | | |
| vertical after | (20) | | | |
| 20 s (capillary | | | | |
| liquid | | | | |
| absorption) | | | | |
| Elongation at | DIN EN ISO | 1.2% | 9.4% | 7.7% |
| 5N along 1 | 9073-03 | | | |
| inch of | | | | |
| sample width | | | | |

The test results show that the method according to the invention can significantly improve the cushioning effect (compression hardness), the ability to absorb liquid (quantitative absorption) and the ability to store (run in) compared to the state of the art.

1. A method for manufacturing an absorption and distribution nonwoven fabric made of staple fibers and absorbent material for personal hygiene products, wherein the nonwoven fabric is composed of thermoplastic, synthetic staple fibers as support fibers, wherein the support fibers are homo- or bi-component, thermoplastic polymer fibers comprising fusible constituents, staple fibers of thermoplastic and/or duroplastic polymers as distribution fibers and absorbent material composed of regenerated cellulose, wherein the nonwoven fabric is mechanically bonded and then bonded by means of subsequent thermal activation, wherein
the nonwoven fabric is composed of 5 to 35 wt. % of support fibers, 30 to 90 wt. % of distribution fibers and 5 to 35 wt. % of absorbent material, and
the mechanical bonding is carried out by means of needling.

2. The method according to claim 1, wherein the needling is carried out with at least 200 active barbs/cm² of nonwoven fabric, preferably with 200 to 1000 active barbs/cm² nonwoven fabric, more preferably with 300 to 600 active barbs/cm² nonwoven fabric, most preferably with 400 to 500 active barbs/cm² nonwoven fabric.

3. The method according to claim 2, wherein individual needles used for needling comprise between 1 and 9, preferably between 3 and 6, barbs and/or a diameter between 30 and 46 gauge, preferably between 38 and 40 gauge, and a length of 2 to 3 inches.

4. The method according to claim 2, wherein the needling is carried out with at least two, preferably four, needle boards, which penetrate the incoming nonwoven alternately on both sides, wherein the needle boards preferably comprise a needle density of 12,000 to 80,000 needles/m of working width, particularly preferably a needle density of 20,000 to 50,000 needles/m of working width.

5. The method according to claim 1, wherein the fibers of the nonwoven fabric are homogeneously mixed with one another before the mechanical bonding and are formed into a fibrous web by means of a carding process.

6. The method according to claim 5, wherein the fibrous web is laid down in the longitudinal direction.

7. The method according to claim 5, wherein the fibrous web is laid down as a layered structure comprising a first layer and a second layer, wherein the first layer is laid in the longitudinal direction and the second layer is laid in the transversal direction or with an isotropic orientation and is laid on or under the first layer.

8. The method according to claim 7, wherein the layered structure comprises two first layers, wherein the second layer is laid between the two first layers.

9. The method according to claim 8, wherein the two first layers each comprise 25-40% of the weight per unit area and the second layer comprises 20-50% of the weight per unit area.

10. The method according to claim 1, wherein the nonwoven fabric, after mechanical and thermal bonding, comprises a material thickness per weight per unit area of at least 0.20 mm/10 g/m², preferably at least 0.25 mm/10 g/m².

11. The method according to claim 1, wherein the thermal activation is carried out by means of hot-air bonding, wherein the nonwoven fabric is preferably heated during the hot-air bonding for at least 20 seconds to a temperature above the melting point of fusible constituents of the support fibers.

12. Absorption and distribution nonwoven fabric produced by a method according to claim 1.

13. The Absorption and distribution nonwoven fabric according to claim 12, wherein the nonwoven fabric comprises a weight per unit area of 40 to 150 g/m², preferably between 50 and 100 g/m².

14. An apparatus for carrying out a method according to claim 1.
